(19) United States
(12) Patent Application Publication
(10) Pub. No.: US 2016/0367950 A1
(43) Pub. Date: Dec. 22, 2016

(54) A METHOD FOR PRODUCING NANOSTRUCTURED OR MICROSTRUCTURED MATERIALS AND A DEVICE FOR THEIR PRODUCTION

(71) Applicant: CESKE VYSOKE UCENI TECHNICKE V PRAZE, Praha 6 (CZ)

(72) Inventors: Milos BERAN, Praha 5 (CZ); Frantisek TOMAN, Praha 8 (CZ); Josef DRAHORAD, Praha 8 (CZ); Jiri HOVORKA, Poricany (CZ); Zdenek HUSEK, Praha 6 (CZ)

(73) Assignee: CESKE VYSOKE UCENI TECHNICKE V PRAZE, Praha 6 (CZ)

(21) Appl. No.: 14/787,599

(22) PCT Filed: May 15, 2015
(86) PCT No.: PCT/CZ2015/000043

§ 371 Date: Oct. 28, 2015

(51) Int. Cl. B01F 3/04; B03C 3/00; B01D 46/00; B01D 1/18; F26B 3/12; B01D 1/20; C08J 3/12; C07K 14/77; A61K 35/747; A61K 35/745; A61K 9/50; C01D 3/06; C12N 1/20; C12N 9/02; B01D 45/16

(52) U.S. Cl. CPC B01F 3/04021; B01D 45/16; B03C 3/00; B01D 46/00; B01D 1/18; F26B 3/12; B01D 1/20; C08J 3/12; C07K 14/77; A61K 35/747; A61K 35/745; A61K 9/5047; A61K 9/5089; C01D 3/06; C12N 1/20; C12N 9/0095; C12Y 118/06001; C08J 2329/04; C12N 2500/02

(57) ABSTRACT

A device for producing nanostructured or microstructured with materials comprises a chamber in which a hollow shaft is assembled, at least one disc provided with an expansion gap. The hollow shaft has openings which connect the inner space of the hollow shaft with the expansion gap.

A solution, emulsion or liquid suspension of substances or microorganisms optionally saturated with a gas, liquefied gas or supercritical liquid, is fed into an inner space of a disc through a hollow shaft. By means of the combination of a centrifugal force and fluid pressure occurs the outlet of the liquid through an expansion gap, to form microscopic droplets. The microscopic droplets are subsequently disintegrated by expansion of the gas to form an aerosol. The aerosol is subsequently dried by a drying gas stream to form solids.

### TECHNICAL FIELD

This invention relates to nanostructured or microstructured materials and devices for their production.

### BACKGROUND ART

Currently there are numerous methods for producing nanostructured and microstructured materials known, as well as corresponding devices for their production. One of them is a method, the solution of which consists in that the solution for the production of these materials is stored in a separate container and transported by a pump via a pipeline to a mixing chamber where it is mixed with pressurized carbon dioxide, which is also separately conveyed by a pump into the mixing chamber. From the mixing chamber the solution is conveyed, directly into a nozzle. In some cases, the mixing chamber is even omitted, and the mixing of gas and the solution of the material occurs only in the actual nozzle. Said processes are characterized by frequent clogging of nozzles, resulting in disruption of the production and limiting the productivity.

### SUMMARY OF THE INVENTION

Said disadvantages of the method of producing nanostructured and microstructured materials and devices for performing this method can largely be removed by a solution according to the invention, the principle of which consists in that a solution, emulsion or liquid suspension of one substance or a mixture of substances or microorganisms optionally saturated with a gas, liquefied gas or supercritical liquid, is fed into a disc interior through a hollow shaft, where by means of the combination of centrifugal forces and fluid pressure the outlet of the liquid through an expansion gap is activated to form microscopic droplets. The microscopic droplets are subsequently secondarily disintegrated in a drying chamber by expansion of the gas comprised therein to smaller droplets forming an aerosol. The aerosol is subsequently dried by a drying gas stream to form solids. In special cases, when drying some polymers under certain conditions, microfibers or nanofibers can be created instead of corpuscular forms.

The solution, emulsion or liquid suspension of one substance or a mixture of substances or microorganisms optionally saturated with a gas, liquefied gas or supercritical liquid, is pumped into an inner space of a disc under a pressure from 10 to 400 bar and passes through the expansion gap into the drying chamber, whereby the pressure in the drying chamber is equal to the atmospheric pressure or it is lower than the pressure of the saturated solution. Into the chamber, a drying gas with defined properties is blown. The drying gas can be air or nitrogen at a temperature from 20 to 200° C. having defined moisture.

The created nanostructures or microstructures are separated in the solid state from the stream of drying gas and the gas serving to the liquid saturation using a filter, cyclone, or electrically charged collector.

In the case of a dual rotating disc, the size of the expansion gap is created by deformation of at least one part of the disc depending on the pressure of the liquid medium inside the disk and the pressure generated by the pressure element.

The gas, liquefied gas or supercritical liquid can be in a preferred embodiment carbon dioxide.

The subject matter of a device for producing nanostructured and microstructured materials according to the invention consists in that it comprises a chamber in which a hollow shaft is assembled on which at least one disc provided with the expansion gap is mounted, wherein the hollow shaft has openings which connect the inner space of the hollow shaft with the expansion gap. The chamber may additionally be provided with an independent feed nozzle.

It is preferred that at least one disc is rotating and is formed by two successive parts, wherein between the upper part and the lower part the expansion gap is formed around the circumference. It is preferred that the expansion gap is formed around the whole circumference of at least one disc.

At least one of the parts of the rotating disc is provided with a pressure element. It is preferred that the pressure element is a presser nut. At least one part of the disc or a rotating disc may be of frustoconical shape.

The hollow shaft is connected to a rotary unit that connects the stationary part of the device with the hollow shaft and allows the entry of liquid from the stationary part of the device.

The invention is based on the use of the disc, which is provided with outlet nozzles or the internal space with an expansion gap, into which the liquid is fed through the hollow rotating shaft, if the disc is composed of two parts, the expansion gap opens by means of stretching at least one part or the disc through a material deformation in the width from 1 to 500 micrometers at a over-pressure in the range of 10 to 400 bar, which is controlled by a pressure element, for example a nut.

The pressure rotating disc combines liquid spraying by means of nozzles or the expansion gap due to the centrifugal force and over-pressure of the liquid in the disc inner space with a secondary atomization caused by the subsequent rapid expansion of carbon dioxide from the microdroplets in the drying chamber resulting in the formation of a very fine aerosol.

In comparison with devices using static nozzles, the new presented technical solution allows a significant increase in flow rate of the solution, drying rate and thus the productivity of the whole production. The device is particularly suitable for a quick and gentle drying thermolabile molecules or microorganisms while retaining their activities and vitality.

### BRIEF DESCRIPTION OF DRAWINGS

An exemplary embodiment of the device for producing nanostructured or microstructured materials is shown in the accompanying drawings, wherein

FIG. 1 shows an overall diagram of the entire device,

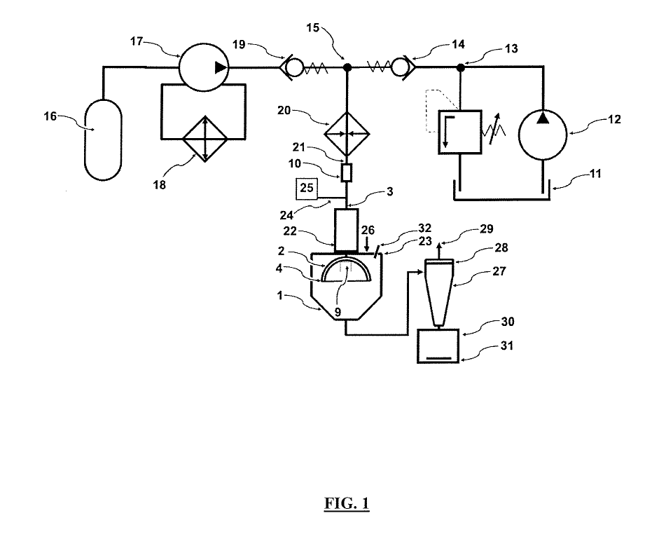

FIG. 2 shows the hollow shaft with the disc in an axonometric view and a partial longitudinal section and

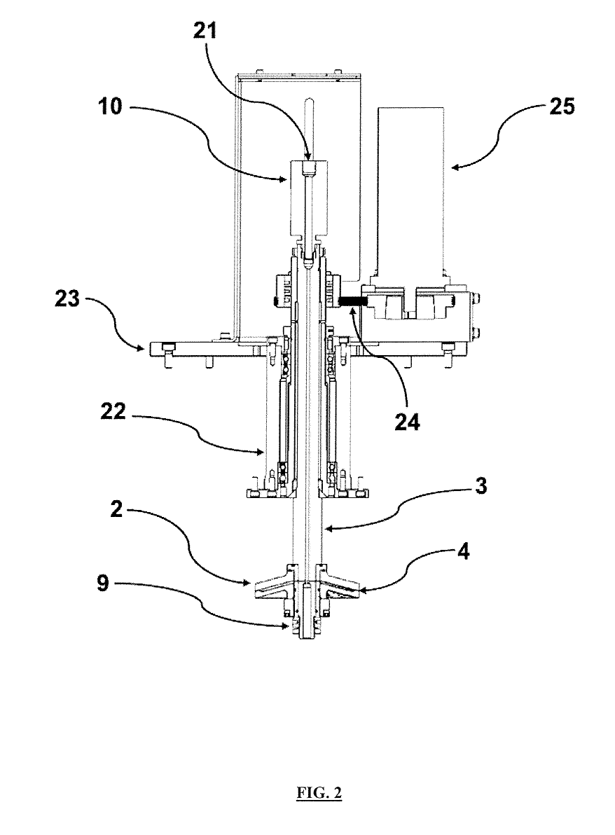

FIG. 3 shows a specific embodiment of the disc according to the invention.

### DETAILED DESCRIPTION OF THE INVENTION

### EXAMPLE 1

### Drying NaCl

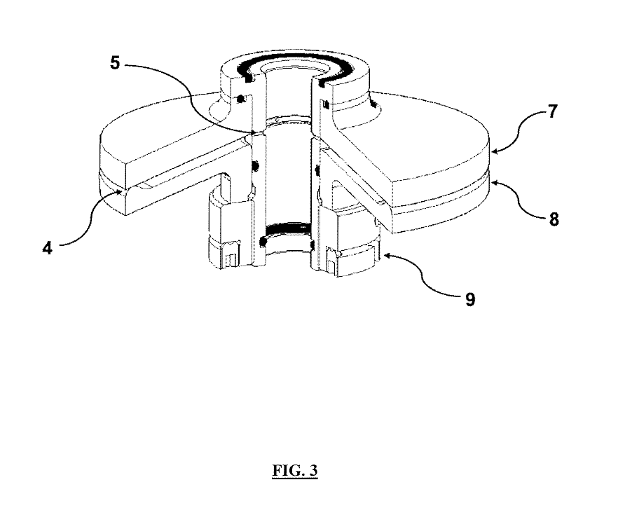

Sodium chloride was selected as model inorganic salt. It was prepared 5 litres 10% (wt./wt.) of NaCl solution. The solution was pumped from a reservoir 11 of a liquid by a high pressure pump 12 at a flow rate of 60 ml/min, through a safety valve 13 and a first check valve 14 into a mixing chamber 15. Simultaneously, carbon dioxide was pumped from a pressure vessel 16 by a pump 17 for carbon dioxide, equipped with a condenser 18, through a second check valve 19 into the mixing chamber 15. The sodium chloride solution, which was in the mixing chamber 15 saturated with carbon dioxide, passed through a heater 20 and a fluid inlet 21 to a rotary unit 10 from which advanced further into an inner space 6 of a hollow shaft 3 disposed in a tube 22 in a base frame 23 of a drying chamber 1. From the inner space 6 of the hollow shaft 3, the solution saturated with carbon dioxide entered through holes 5 of the hollow shaft 3 into the internal space of the disc 2 between its upper part 7 and the lower part 8. The disc 2 of a conical shape was used with a diameter of 120 mm, with a pressure element 9 in the form of a nut as can be seen from FIG. 3. The pressure of the pressure nut was gradually changed so that the opening of the expansion gap 4 occurred at a pressure in the range of 10 to 400 bar. The rotating disc 2 with the hollow shaft 3 was rotated via embedded gears 24 by means of a driving motor 25 at a velocity from 0 to 10,000 rpm. Through the base frame 23, drying air preheated to a temperature of 35° C. was blown from a source of drying gas 26, which was formed by a compressor and a heater, into the drying chamber lat a velocity of 0.8 m³/min. Microscopic droplets were subsequently in the drying chamber 1 secondarily disintegrated by expansion of carbon dioxide escaping from the saturated liquid into smaller droplets, resulting in a very fine aerosol

This aerosol was dried in the drying chamber 1 in a stream of preheated air. The resulting sodium chloride microcrystals were separated from the stream of drying air and carbon dioxide in a cyclone 27 for the separation of particles. The upper part of the cyclone under the outlet 29 of the drying gas was equipped with a permeable filtering membrane 28 with a nanofiber layer, and the dried microcrystals of sodium chloride were collected in a collecting vessel 30 for the dried material. The effectiveness of the separation of sodium chloride particles was greater when the collecting container 30 had been equipped with an electrically charged collector 31.

Different drying conditions of sodium chloride were tested. In one case, the drying was carried out without rotating the disc 2. The primary atomization of the sodium chloride solution was here limited to the spraying in the narrow expansion gap 4 due to the overpressure in the inner space of the disc 2 only, without using a centrifugal force, as in the case of spraying on a nozzle; secondarily, disintegration of the originated microdroplets due to the expansion of carbon dioxide occurred, causing the production of even smaller droplets, likewise, the influence of the speed of the rotation of the disk 2 at a constant, flow of sodium chloride and carbon dioxide through the system upon the size of the originated microcrystals of sodium chloride was tested. Furthermore, it was tested how the size of the microcrystals produced was affected by changes in pressure within the inner space of the disc 2. The pressure in the interior of the disc 2 was controlled by tightening or releasing the pressure member 9. Drying NaCl was also realized at zero flow of carbon dioxide, merely by the primary atomization due to the centrifugal force generated by the disc 2 rotation and the over-pressure of the liquid inside the inner space of the disc 2. In this case, no secondary atomization due to the expansion of carbon dioxide from the resulting microdroplets took place. Finally, the possibility of placing two rotating discs 2 over each other on the hollow shaft 3 or on two independent hollow shafts 3 was tested.

In the case of drying without rotation of the disc 2, the size distribution of the microcrystals, expressed as the length of the wall of the cubic microcrystals, was in the range from 2 to 8 microns, depending on the pressure of the inner space of the disc 2, which was regulated, by tightening the pressure element 9 in the range of 10 to 400 bar. The size of the microcrystals produced diminished with increasing the pressure in the inner space of the disc (2). At the zero flow of the carbon dioxide, the size distribution of the microcrystals ranged from 30-150 microns depending on. the rotation speed, which ranged between 100 and 10,000 revolutions per minute. With increasing the rotation speed of the disc 2, the size of the microcrystals produced decreased. At a constant flow of the sodium chloride solution and carbon, dioxide through the system, the size distribution of the microcrystals was in the range from 0.5 to 3 microns, depending on the rotation speed of the disc 2 and the pressure in the inner space of the disc 2. The size of the microcrystals produced diminished again with the increasing pressure in the inner space of the disc 2, and the increasing rotations of the disc 2.

Yields of sodium chloride ranged in all experiments between 80 to 95%. Losses of sodium chloride were due to sticking thereof on the wails and in the pipes of the drying chamber 1. It has been demonstrated that two-stage atomization realized by a combination of the primary atomization by means of the centrifugal force generated by the rotation of the disk 2 and the liquid over-pressure in the inner space of the disk 2, and the secondary atomization by means of the expansion of carbon dioxide from the resulting microdroplets, allows to reduce the size of the microcrystals of sodium chloride. Effects of the primary and secondary atomization therefore summarize and allow the production of smaller dry particles, than if these methods of primary and secondary atomization were used separately. Placing multiple discs 2 on the same hollow shaft 3 or on independent hollow shafts 3 in the same drying chamber 1 allows the increase of the drying speed.

### EXAMPLE 2

### Drying Polyvinyl Alcohol

Polyvinyl alcohol was chosen as a model spinnable polymer. For experiments, a commercial solution of polyvinyl alcohol Sloviol R16, 16% (wt./wt.) of solids (Fichema) was used. The arrangement, conditions and apparatus of the experiment were the same as in Example 1. The flow of the polyvinyl alcohol solution was 70 ml/min. Due to the centrifugal forces, in the expansion gap 4 of the rotating disc 2, the formation of nanofibers and microfibers took place. The rate of the fibers formation gradually increased in the range of the rotation speed of the disc 2. The pressure in the inner space of the disc 2 had no significant effect upon the formation rate of the fibers. The yields of polyvinyl alcohol in the fibers were in the range 75-90%, depending on conditions, losses were caused by snicking polyvinyl alcohol on the walls and in the pipeline of the drying chamber 1. The fibers were obtained having a diameter in the range 0.1 to 1 micrometer, depending on the conditions of the experiment, in a form resembling a fine, dense wool. The fiber diameter decreased with the increasing pressure in the inner space of the disc 2 and with the increasing speed of the disc 2 in the range from 500 to 3000 rpm. Upon further increasing the speed of rotation of the disc 2, there occurred already a prevalent formation of microdroplets and the formation of irregularly shaped particles.

### EXAMPLE 3

### Drying Ovalbumine as Model Proteins

Egg white ovalbumine (Sigma-Aldrich) was chosen as a model protein. The arrangement, conditions and apparatus of the experiment were the same as in Example 1. In distilled water, a solution comprising 5% (wt./wt.) ovalbumine and 5% (wt./wt.) trehalose (Fluka) was prepared. Trehalose has been used as a stabilizing agent. The flow of the ovalbumine solution was 90 ml/min. Spherical particles were obtained having a diameter ranging from 0.4 to 2 microns depending on the experiment conditions. The particle diameter decreased with the increasing pressure in the inner space of the disc 2 and with the increasing speed of the disc 2. In an alternative embodiment, a disk 2 having the diameter of 120 mm, with ten outlet nozzles over the circumference was used for the primary atomization of the ovalbumine solution instead of the disc 2 having the expansion gap. The diameter of the individual outlet nozzles was 100 micrometers. In this case, while maintaining the same conditions, the spherical particle size was in the range of 1-3 micrometers.

### EXAMPLE 4

Drying Heterocysts Isolated from Cyanobacterias and Enzyme Nitrogenase

Drying heterocysts was chosen as a model of gentle drying living cells while preserving their vitality. Drying of the enzyme nitrogenase isolated from heterocysts illustrates the possibility of gentle drying enzymes while retaining their biological activity and the possibility of drying under anaerobic conditions. Heterocysts are specialized cells of some filamentous cyanobacterias with a thin cell wall of a light yellow colour. Their function is to fix nitrogen from the air in case of deficiency of other forms of this element. Keterocysts use for the fixation of atmospheric oxygen the enzyme nitrogenase that is inactivated by oxygen. Keterocysts must create microanaerobic environment. Keterocysts were isolated from fibres of cyanobacterias Cyanobacterium Anabaena sp., strain CA (ATCC 330-17) by a procedure disclosed in the publication by Smith R. L. et al. (R. L, Smith, D. Kumar, Z. Xiankong F, R, Tabita, and C, Van Baalen 1985, K2, N2 and O2 metabolism by isolated heterocysts from Anabaena sp. Strain CA. J. Bacteriol. 162: 565-570). The metabolic activity of isolated heterocysts was measured by the reduction of acetylene in anaerobic conditions using the methodology described by Kumar A. et al. (A. Kumar, F. R. Tabita, and C, van Baalen, 1983. High endogenous nitrogenase activity in isolated heterocysts of Anabaena sp. strain CA after nitrogen starvation. J. Bacteriol. 155 (2): 565-570). A part of heterocysts obtained was used to isolate the enzyme nitrogenase (EC1.7.99.2) by a method described by Song S.-D. et al. (Song S.-D., A. Hartmann, and R H Burris. 1985, Purification and Properties of the Nitrogenase of Azospirillum amazonense, J. Bacteriol. 164 (3): 1271-1277). Activity of the isolated nitrogenase was again measured by the acetylene reduction under anaerobic conditions as described in the publication Shah V. K. et al. (V. K, Shah, L.C. Davis, and W. J. Brill. 1975. Nitrogenase. VI. Acetylene reduction assay: Dependence of nitrogen fixation estimates on component ratio and acetylene concentration. Biochem. Biophys. Acta 384 (2): 353-359).

The isolated heterocysts and nitrogenase were stored without access of air under a nitrogen atmosphere. Heterocysts were suspended in a physiological saline to the dry matter 6% (wt./wt.). The suspension was maintained in the liquid reservoir 11 under a nitrogen atmosphere. The experimental arrangement and equipment were the same as in Example 1. The flow of the ceil suspension was 80 ml/min. The pressure in the inner space of the disc 2 was set by a presser nut at 60 bar. The drying gas was in this case nitrogen. The source 26 of nitrogen was a large capacity pressure vessel. The flow of nitrogen through the drying chamber 1 was 0.8 m³/min., the temperature of nitrogen entering the drying chamber 1 was 40° C. The dried cell culture was separated from the stream of nitrogen and carbon dioxide in the cyclone 27 and collected in the collecting vessel 30. The product was in a form of a fine powder. The yield of the heterocysts in dry form was more than 30%. The vitality decline of the cell culture was only 4.7%. The decline in metabolic activity, measured as the reduction of acetylene under anaerobic conditions, was not statistically significant.

Nitrogenase was suspended in distilled water to a concentration of 5% (wt./wt.) with the addition of 5% (wt./wt.) sucrose, which served as a stabilizing agent. Nitrogenase was dried under the same conditions as heterocysts. Spherical particles of diameter about 1 micron were obtained. The yield of nitrogenase in the dry form was approximately 80%. Even, in this case the decrease of the enzyme activity was not statistically significant.

### EXAMPLE 5

### Encapsulation of Probiotic Bacteria in Water Suspensions of Cellulose Derivatives

This example was chosen as a demonstration of the possibility to use the device according to the invention for encapsulating compounds or microorganisms. Probiotic microorganisms must meet certain basic requirements in order to bring health benefits to their host. It belongs among these basic requirements that such probiotic microorganisms must be sufficiently resistant to the stomach acidic environment and the action of bile acids in the small intestine. However, by no means all commercially available strains of probiotic microorganisms fully comply with these requirements. One of the often used methods to increase their resistance to these influences is their encapsulation with various materials.

In the first part of the experiment, a suspension containing 0.5 l of the commercial enteric formulation of ethyl cellulose in the nanoparticulate form FMC's Aquacoat ECD and 2 l of a similar formulation containing cellulose acetate phthaiate FMC's Aquacoat CPD, 2 kg of the microbial preparation BA (1.10⁹ CFU/g) (Milcom), containing the probiotic strains of genera Lactobacillus acidophilus and Bifidobacterium bifidum freeze-dried with powdered milk, 200 g of the prebiotic preparation inulin Frutafit HP and 5 l of distilled water. The experimental arrangement and equipment were the same as in Example 1. The drying gas was preheated air to a temperature of 35° C., which was blown in the drying chamber (1) at the velocity of 0.8 m³/min. from a source (26) consisting of a compressor and a heater. The flow of the dried suspension was 75 ml/min. The dried cell culture was separated from the stream of drying air and carbon dioxide in the cyclone 27 and collected in the collecting vessel 30. The product was in the form of a fine powder. Bacteria were encapsulated inside the particles of cellulose derivatives. The particles were irregularly shaped. The particle size distribution was in the range 4-7 microns. The yield of the dry matter of the suspension was about 80%. The standard methods for microbiological analysis revealed that there was no statistically significant decrease in vitality of the original bacterial culture. Microbiological tests confirmed a significant protective effect of encapsulating against the simulated acidic environment of the stomach and the action of bile acids.

In the second part of the experiment, a suspension containing 0.5 l of the commercial enteric formulation of ethyl cellulose in the nanoparticulate form FMC's Aquacoat BCD and 2 l of a similar formulation containing cellulose acetate phthaiate FMC's Aquacoat CPD in 3.8 l of distilled water. In addition to this, a bacterial, suspension was prepared containing 2 kg of the microbial preparation EA (1.10⁹ CFU/g) (Milcom), and 200 g of the prebiotic preparation inulin Frutafit H. Both suspensions were simultaneously injected into the drying chamber 1 by two rotating disks 2 on independent hollow shafts 3, or by a combination of the rotating disc and independent feed nozzle 32. The drying gas was again preheated air to a temperature of 35° C., which was blown into the drying chamber 1 at the velocity of 0.8 m³/min, from the source 26 composed of a compressor and a heater. The flow of the dried suspension through each rotating disc or a nozzle was identically 75 ml/rain. The dried cell culture was separated from the stream of drying air and carbon dioxide in the cyclone 27 and collected in the collecting vessel 30. The product was in the form of a fine powder. Bacteria were encapsulated, inside the particles of cellulose derivatives. The particles were irregularly shaped. The particle size distribution was in the range 3-6 microns. The yield of the dry matter of the suspension was about 85%. In this example, there was also no statistically significant decrease in vitality of the original bacterial culture. Microbiological tests confirmed, again a significant protective effect of encapsulating against the simulated acidic environment of the stomach and the action of bile acids.

The combination of two different discs 2 on independent hollow shafts 3 or a combination of the disc 2 with the independent a feed nozzle 32 allows the combination of both the atomization and drying of two different liquids—solutions, emulsions or suspensions simultaneously in the same drying chamber 1. The dried material is produced by the combination and interaction of the components of these two different liquids in the drying chamber.

### INDUSTRIAL APPLICABILITY

This invention relates to a process of production of nanostructured or microstructured materials and a device for their production. In comparison with devices using static nozzles, the new presented technical solution allows a significant increase in the flow of the solution, the drying speed and thus the productivity of the whole production. The device is especially suitable for quick gentle drying thermolabile molecules or microorganisms while retaining their activities and vitality.

### LIST OF REFERENCE NUMBERS

1 —chamber
2 —disc
3 —hollow shaft
4—expansion gap
5—opening
6 —inner space
7 —upper part
8 —lower part
9 —pressure element
10—rotary unit
11—magazine
12—high-pressure pump
13—safety valve
14—first check valve
15—mixing chamber
16—pressure vessel
17—pump
18—cooler
19—second back valve
20—heater
21—input
22—tube
23—base frame
24—embedded gears
25—drive motor
26—source
27—cyclone
28—filtration membrane

29 —output

30 —collecting vessel

31 —electrically charged collector

32 —independent feed nozzle

1. A method for producing nanostructured or microstructured materials, wherein a solution, emulsion or a liquid suspension of one substance or mixtures of substances or: microorganisms optionally saturated with a gas, liquefied gas or supercritical, fluid is fed through a hollow shaft into an inner space of a disk fitted with outlet nozzles or an expansion gap, wherein the combination of a centrifugal force and/or pressure leads to atomise the liquid to form microscopic droplets.

2. The method for producing microstructured or nanostructured materials as defined in claim 1, wherein the microscopic droplets in the drying chamber are secondarily disintegrated by expansion of a gas escaping from the saturated liquid into smaller droplets, resulting in a very fine aerosol.

3. The method for producing microstructured or nanostructured materials as defined in claim 1, wherein a solution, emulsion or a liquid suspension of one substance or mixtures of substances or microorganisms optionally saturated with a gas, liquefied gas or supercritical fluid, is fed into nozzles or an expansion gap of a rotating disc under a pressure from 10 to 400 bar, whereby the pressure in the drying chamber is equal to the atmospheric pressure or it is here lower than the pressure in the inner space of the disc.

4. The method for producing microstructured or nanostructured materials as defined in claim 2, wherein the aerosol is subsequently dried by a gas stream.

5. The method tor producing microstructured or nanostructured materials as defined in claim 4, wherein the drying gas is air or oxygen at a temperature of 20 to 200 degrees Celsius.

6. The method for producing microstructured or nanostructured materials as defined in claim 1 wherein the formed solid nanostructures or microstructures are separated from the gas mixture passing out from the chamber using a filter, cyclone, or electrically charged collector.

7. The method for producing microstructured or nanostructured materials as defined in claim 1 wherein in the case of a dual rotating disc, the size of the expansion gap is created by a deformation of one part of the disc in dependence on the pressure of the blown medium and the pressure generated by a pressure element.

8. The method for producing microstructured or nanostructured materials as defined in claim 1, wherein the gas, liqufied gas or supercritical fluid is carbon dioxide.

9. A device for producing nanostructured or microstructured materials, wherein it comprises a chamber in which a hollow shaft is assembled on which at least one disc provided with the inner space having an expansion gap is mounted, wherein the hollow shaft has openings which connect the inner space of the hollow shaft with the inner space of the disc.

10. The device for producing nanostructured or microstructured materials as defined in claim 9, wherein at least one disc is rotating and is formed by two successive parts, where between the upper part and the lower part an expansion gap is formed around the circumference thereof.

11. The device for producing nanostructured or microstructured materials as defined in claim 9, wherein at least one of the parts of the rotating disc is fitted with a pressure element.

12. The device for producing nanostructured or microstructured materials as defined in claim 11, wherein the pressure element is a pressure nut.

13. The device for producing nanostructured or microstructured materials as defined in claim 8, wherein the expansion gap is created around the whole circumference of at least one disc.

14. The device for producing nanostructured or microstructured materials as defined in claim connected to a rotary unit that connects the stationary part of the device with the hollow shaft and allows the entry of the liquid from the stationary part of the device.

15. The device for producing nanostructured or microstructured materials as defined in claim 9 wherein at least one part of the disc or the rotating disc is of frustoconical shape.

16. The device for producing nanostructured or microstructured materials as defined in claim 9, wherein the chamber is provided with an independent feed nozzle.
